(19) United States
(12) Patent Application Publication
(10) Pub. No.: US 2012/0152241 A1
(43) Pub. Date: Jun. 21, 2012

(54) Breathing Apparatus

(76) Inventor: Emily Pettitt, East Sussex (GB)

(21) Appl. No.: 13/392,691

(22) PCT Filed: Aug. 31, 2010
(86) PCT No.: PCT/GB10/01638

§ 371 Date: Feb. 27, 2012

(30) Foreign Application Priority Data
Sep. 10, 2009 (GB) 0915818.9

(51) Int. Cl. A61M 16/06

(52) U.S. Cl. 128/201.23

(57) ABSTRACT

Patient breathing apparatus comprises a hood having a front single-layer, transparent sheet and a rear two-layer section. An inflatable cuff extends around the lower end of the hood and is connected to a recirculating heliox gas supply unit. The cuff has perforations in its upper surface around the front part of the hood so that the gas supplied to inflate the cuff flows into the inside of the hood to provide at least a part of the breathing gas. The inner layer of the rear section is permeable so that the gas can flow from the inside of the hood into a space between the two layers and there to a gas outlet. A hose connects the gas outlet to an inlet of the gas supply for recirculation.

This invention relates to breathing apparatus of the kind including an enclosure arranged to enclose the entire head of the user and a supply of breathing gas to the enclosure.

The invention is more particularly concerned with apparatus for providing breathing gas to patients having respiratory problems.

It is known that patients with respiratory problems can benefit from a gas mixture of oxygen and helium, the latter gas reducing the patient's work of breathing. Helium, however, is relatively scarce and expensive so conventional ventilation techniques where exhaled gas is exhausted to atmosphere are very wasteful and costly. The high cost is such that treatment with helium can only usually be provided to patients with severe respiratory problems.

Various ways are used to supply breathing gas to a patient, such as face masks, nasal masks or mouthpieces. These can be uncomfortable in prolonged use, often causing damage to the skin around the mouth or nose; they usually prevent the patient talking and often prevent make it difficult to wear spectacles. Alternatively, a hood may be used to enclose the entire head. One problem with hoods lies in the difficulty of making an effective seal with the patient. This may not be a problem where low cost gases are used and leakage is not of concern but when an expensive gas, such as a helium mixture is used, it is necessary to minimise leakage so that the maximum proportion of gas can be recovered and reduce the entrainment of ambient air, which would otherwise dilute the breathing gas.

It is an object of the present invention to provide alternative breathing apparatus.

According to one aspect of the present invention there is provided breathing apparatus of the above-specified kind, characterised in that the lower end of the enclosure is provided with an inflatable cuff that is inflated by gas to seal the enclosure about the user's neck.

The inflatable cuff preferably also provides a path by which a part at least of the breathing gas is supplied to the inside of the enclosure such that a part at least of the breathing gas supplied to the enclosure also inflates the cuff The cuff is preferably permeable to breathing gas on a side exposed to the inside of the enclosure. The cuff may be permeable to gas only along a front part of the cuff located towards the front of the user's head. The cuff may be permeable by virtue of perforations in the cuff on the surface facing the interior of the enclosure. A part at least of the enclosure may be formed of two layers, an outer layer substantially impervious to the breathing gas and an inner layer permeable to the breathing gas, the two layers being separated from one another by a channel connected to gas means. The two layers may be separated by a plurality of spacers. The part formed of two layers preferably extends around only a rear part of the enclosure located towards the rear of the patient's head. A part of the apparatus towards the front of the user's head is preferably provided by a single layer of a transparent material. The two-layer part of the enclosure preferably provides an outlet for gas from the enclosure. The apparatus preferably includes a gas supply including a source of helium gas and may be a recirculating gas supply.

According to another aspect of the present invention there is provided breathing apparatus including an enclosure arranged to enclose the entire head of the user, characterised in that a part at least of the enclosure is formed of two layers, an outer layer substantially impervious to breathing gas and an inner layer permeable to breathing gas, and that the two layers are separated from one another by a channel connected to gas means.

The part formed of two layers preferably extends around only a rear part of the enclosure located towards the rear of the user's head. The two-layer part of the enclosure preferably provides an outlet for gas from the enclosure.

Breathing apparatus in the form of a hood according to the present invention will now be described, by way of example, with reference to the accompanying drawings, in which:

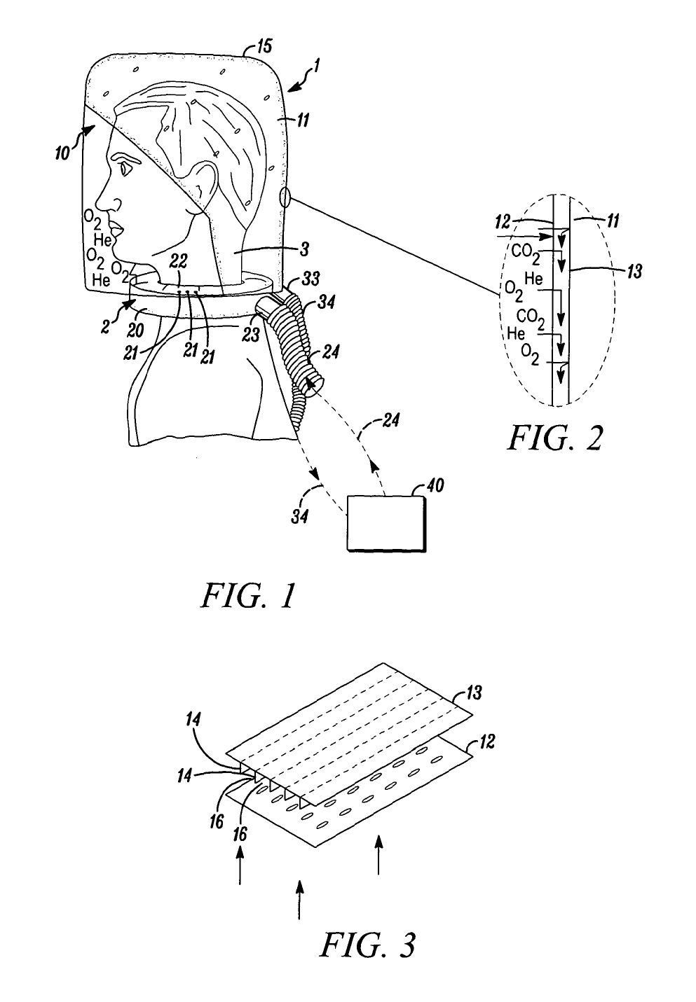

FIG. 1 is a side elevation view of hood;

FIG. 2 is an enlarged cross-sectional side elevation view of the hood material; and

FIG. 3 is an enlarged exploded perspective detail of a part of the hood material.

The hood or enclosure has an upper part 1 that encloses the head of the user and a lower part 2 that seals around the user's neck 3.

The upper part 1 is in two sections, namely a forward-facing front section 10 and a rear section 11. The front section 10 is a single sheet of an optically transparent plastics material and is preferably flexible but self-supporting. The front section 10 extends laterally around the front and side of the user's head to the region of the user's ears and vertically to the region of the user's brow, being separated from the top of the hood by a part of the rear section 11. The front section 10 enables unrestricted visibility by the user and enables clinicians to view the patient without obstruction.

The rear section 11 is opaque or translucent, being formed of an inner layer 12 and an outer layer 13. The inner layer 12 is gas-permeable and may be made of a fabric, non-woven or plastics material. Its permeability may be due to a porous nature of the material or to perforations formed through the material. The outer layer 13 is of an impermeable material such as a plastics film and is formed on its inner surface with spacers in the form of closely-spaced parallel ribs 14 extending vertically on the side surfaces of the hood and from front to back on its upper surface 15. The ribs 14 abut the inside surface of the permeable layer 12 and serve to maintain a small separation between the two layers defining parallel gas channels 16 between the ribs. The ribs could be formed instead on the inner layer, or on both layers. Alternative spacers could be used such as an array of pips or the like.

The lower part 2 of the hood includes an inflatable, toroidal or annular cuff 20 arranged to seal around the user's neck 3. The inflatable nature of the cuff 20 means that it automatically accommodates variations in patient anatomy and resists twisting and shifting of the hood. It is not essential that the cuff be a complete circle since it could have a horseshoe shape and extend around the front of the patient with some other form of seal, such as a foam pad, provided at the rear. The cuff 20 is formed with perforations 21 on its upper, inner surface 22, inside the upper part 1, around the front two thirds of its circumference. The lower surface and rear part of its upper surface 22 is not perforated and is impervious to gas, so that gas flowing out of the perforations is confined to the region of the front of the patient's head. A gas inlet coupling 23 opens into the cuff 20 towards its rear and this connects via a hose 24 with a supply of a helium oxygen mixture provided by a rebreather unit 40. Gas from this unit 40 flows via the hose 24 to inflate the cuff 20 and seal the hood around the neck 3. At the same time, the gas supplied passes to the interior of the hood via the perforations 21 in the cuff 20 to provide a breathing mixture of helium and oxygen for the patient. It may be that insufficient breathing gas can be supplied via the cuff perforations 21, in which case, an additional parallel inlet path (not shown) to the hood could be provided directly from the gas inlet coupling 23.

A gas outlet coupling 33 and hose 34 connects to the rear section 11 of the wall of the hood, opening into the channels 16 between the two layers 12 and 13. The hose 34 extends to the inlet of the rebreather unit 40, which includes a scrubber (not shown) or other means for removing carbon dioxide from exhaled breath. After the carbon dioxide has been removed the remaining gas is mixed with additional helium and oxygen and supplied back to the hood via the hose 24 and coupling 23.

In use, therefore, a helium oxygen gas mixture is supplied to the interior of the hood, via the inflated sealing cuff 22 and in the region of the front of the head. Exhaled gas, with elevated carbon dioxide levels, is pushed rearwardly in the hood by the incoming gas and flows to the volume bounded by the rear section 11. This gas flows through the inner layer 12 into the space between the two layers 12 and 13 and, from there, flows down the channels 16 to the gas outlet coupling 33 from where it flows to the rebreather unit 40. The rebreather unit 40 may include a pump to apply a slight negative pressure to the hose 34 so that a negative pressure is created in the channels 16 to draw gas into the space between the layers.

By flowing gas into the hood via the inflatable cuff 20, and allowing gas to flow out of the hood over a large area provided by its permeable rear section 11 a very even distribution of gas within the hood can be produced. The cuff 20 helps create an effective, comfortable seal, minimising escape of gas, which is of particular importance when helium or other expensive gases are used.

The hood, gas supply unit or associated tubing could include a safety valve (not shown) to prevent asphyxiation by opening to atmosphere if pressure from the supply should fall below a set level. A safety valve could also be provided to prevent excess pressure, opening to vent gas in the hood to atmosphere if pressure should rise above a different set level.

1-15. (canceled)

16. Breathing apparatus including an enclosure-and a supply of breathing gas to the enclosure, the enclosure being arranged to enclose the entire head of the user and having a lower end provided with an inflatable cuff that is inflated by gas from said supply to seal the enclosure about the user's neck, characterized in that the inflatable cuff provides a path by which a part at least of the breathing gas is supplied to the enclosure such that a part at least of the breathing gas supplied to the enclosure also inflates the cuff.

17. Breathing apparatus according to claim 16, characterized in that the cuff is permeable to gas on a side exposed to the inside of the enclosure.

18. Breathing apparatus according to claim 17, characterized in that the cuff is permeable to gas only along a front part of the cuff located towards the front of the user's head.

19. Breathing apparatus according to claim 17, characterized in that the cuff is permeable by virtue of perforations in the cuff on the surface facing the interior of the enclosure.

20. Breathing apparatus according to claim 16, characterized in that a part at least of the enclosure is formed of two layers including an outer layer substantially impervious to the breathing gas and an inner layer permeable to the breathing gas, and that the two layers are separated from one another by a channel connected to a gas path.

21. Breathing apparatus according to claim 20, characterized in that the two layers are separated by a plurality of spacers.

22. Breathing apparatus according to claim 20, characterized in that the part formed of two layers extends around only a rear part of the enclosure located towards the rear of the user's head.

23. Breathing apparatus according to claim 22, characterized in that a part of the apparatus towards a front of the user's head is provided by a single layer of a transparent material.

24. Breathing apparatus according to claim 20, characterized in that the two-layer part of the enclosure provides an outlet for gas from the enclosure.

25. Breathing apparatus according to claim 16, characterized in that the gas supply includes a source of helium gas.

26. Breathing apparatus according to claim 16, characterized in that the gas supply is a recirculating gas supply.

27. Breathing apparatus including an enclosure arranged to enclose the entire head of the user, characterized in that a part at least of the enclosure is formed of two layers, an outer layer substantially impervious to breathing gas and an inner layer permeable to breathing gas, and that the two layers are separated from one another by a channel connected to a gas path.

28. Breathing apparatus according to claim 27, characterized in that the part formed of two layers extends around only a rear part of the enclosure located towards the rear of the user's head.

29. Breathing apparatus according to claim 27, characterized in that the two-layer part of the enclosure provides an outlet for gas from the enclosure.
